(19) United States
(12) Patent Application Publication
(10) Pub. No.: US 2005/0209628 A1
(43) Pub. Date: Sep. 22, 2005

(54) System and method for fluid dynamics in a medical procedure

(76) Inventor: Stephen Campbell Brass, Westborough, MA (US)

Correspondence Address: GANZ LAW, P.C., P O BOX 2200, HILLSBORO, OR, 97123, US

(21) Appl. No.: 10/804,783

(22) Filed: Mar. 19, 2004

(51) Int. Cl. A61M 29/00

(52) U.S. Cl. 606/192

(57) ABSTRACT

A prostatic treatment system is provided for applying a transurethral therapy to an organ of a living body with a pressure-pulse-massage via a treatment fluid. The treatment system includes a treatment device to apply therapeutic treatment through pressure-pulse variables including at least one of fluid pressure pulse, pulse amplitude, pulse shape, and pulse frequency. A treatment set is operably coupled to the treatment device to deliver treatment fluid to the treatment device. A flow restrictor is associated with the treatment set for creating a positive pressure in the treatment device. A a control unit independently controls at least one of the pressure-pulse variables.

### FIELD OF THE INVENTION

The present invention generally relates to a method and system for controlling fluid dynamics in a medical procedure. More particularly, the present invention generally relates to a method and apparatus for treating prostatitis.

### BACKGROUND OF THE INVENTION

The most common health-related problems of the prostate are benign prostatic hyperplasia (BPH), prostatitis, and cancer. The prostate is small, walnut-sized gland measuring about 2.5 cm by 1.9 cm by 3.8 cm and a part of the male reproductive system. It is located below the bladder, surrounds the upper portion of the urethra, and lies in front of the rectum.

Prostatitis—inflammation of the prostate—outranks benign prostatic hyperplasia (BHP) and prostate cancer as the most common urologic diagnosis for men less than 50-years old. In fact, unlike BPH, which occurs primarily in older men, prostatitis can occur in both younger (men in age groups of 18-50 (or younger)) and older men (over the age of 50), with the median reported patient age at about 40 years of age. Chronic prostatitis is one of the most common reasons why men visit urologists. At least one report states that 35-50% of men will be affected by prostatitis at some time in their life.

There are several classifications or types of prostatitis, each of which may have different characteristics, manifestations, symptoms, or treatment protocols: Type I: acute bacterial prostatitis; Type II: chronic bacterial prostatitis; Type III: chronic (non-bacterial) prostatitis and/or chronic pelvic pain syndrome (CPPS); and Type IV: asymptomatic inflammatory prostatitis. See Nickel et al., Research Guidelines for Chronic Prostatitis: Consensus Report From the First National Institutes of Health International Prostatitis Collaborative Networlc, Urology, 54(2), pp. 229-233, 230 (1999). Only a small number of reported prostatitis cases are believed to be of the Type I (acute bacterial type), while chronic prostatitis (Types II, III and IV) may affect an estimated 30 million men in the United States.

One of the primary symptoms of prostatitis is chronic urogenital pain. This pain may occur with urination, ejaculation, or in other urogential manifestations. This condition adversely affects the patient's quality-of-life and may be similar to suffering unstable angina, a recent myocardial infarct, or active Crohn's disease. As such, chronic prostatitis is a major health care issue. See J. Curtis Nickel, Prostatitis: Myth and Realities, Urology 51 (3), pp. 362-366 (1998).

Various treatment protocols have been used to attempt to treat prostatitis. Type I often is managed successfully with wide-spectrum antibiotics, however, patients having Type II and III prostatitis have a lower success rate. Other treatments include alpha-blocker therapy, anti-inflammatory agents, lifestyle and diet changes, exercise, modified sexual activity, and supportive psychotherapy. Alternative treatments further include repetitive prostate massage via the rectum, phytotherapy, transurethral microwave thermo therapy, radical transurethral resection of the prostate, radical open prostatectomy, and bladder neck surgery. See Prostatitis: Myths and Realities, p. 365; and Special Report on Prostatitis Initiatives and Future Research; Rev. Urol. 2000; 2(3): 158-166 (Summer 2000). Unfortunately, “[t]he reality of prostatitis treatment is that it results in a dismal cure rate and an unacceptably high relapse or recurrence rate.” Ibid.

More recently, transurethral microwave thermo therapy is suggested as treatment for prostatitis. See, e.g., Choi et al., Clinical experience with transurethral microwave thermotherapy for chronic non-bacterial prostatitis and prostatodynia, Jnl. of Endourology, Vol. 8, pp. 61-64 (1994); Montorsi et al., Is there a role for transrectal microwave hyperthermia of the prostate in the treatment of a bacterial prostatitis and prostatodynia? Prostate, Vol. 22, pp. 139-146, (1993); Nickel et al., Transurethral microwave thermotherapy of nonbacterial prostatitis and prostadynia; initial experience, Urology, Vol. 44, pp. 458-460 (1994); and Nickel et al., Transurethral microwave thermotherapy for nonbacterial prostatitis: a randomized double-blind sham controlled study using new prostatitis specific assessment questionnaires, Journal of Urology, Vol. 155, pp. 1950-1955 (1996). But, transurethral microwave therapy may non-uniformly expose the prostatic tissue to heat from microwave energy, non-targeted tissue may be exposed to microwave energy, the patient must endure use numerous treatments regimes, and success remains uncertain.

Another recent treatment for non-bacterial chronic prostatitis is transurethral needle ablation (TUNA). See e.g., Giannakopoulos, et al., Chronic Nonbacterial Prostatitis and TUNA®: 5 Years Clinical Experience, European Eurology, 37 Supplement, p. 46 (March 2000). Another possible treatment is transurethral radio-frequency method. See Nickel, et al., Transurethral radio frequency hot balloon thermal therapy in chronic nonbacterial prostatitis, Techniques In Urology, Vol. 4, pp. 128-130 (1998).

The aforementioned treatments for prostatitis provide little hope of successfully alleviating the pain experienced by a large percentage of affected individuals. Prostatitis is termed “a waste basket of clinical ignorance” because of the lack of basic epidemiologic knowledge of the disease, the diagnosis, and treatments available. See McNaughton Collins et al., How Common is Prostatitis? A National Survey of Physician Visits, Jnl. Of Urology, Vol. 159, pp. 1224-1228 (April 1998).

A promising treatment for prostatitis combines transurethral therapy with a pressure-pulse-massage. This method incorporates a balloon catheter located in a treatment region of the prostrate. The catheter is filled with a treatment fluid and pressurized slightly. The pressure is modulated about a user-set mean to produce pressure pulses in which the frequency, mean pressure, amplitude, and shape of the pressure pulses are each controlled by a clinician for optimal treatment of prostatitis. This transurethral pressure-pulse-massage treatment method provides minimally invasive methods for treating prostatitis by internally massaging a portion of the prostate. The internal massage is a result of repetitively expanding and contracting a balloon catheter positioned in the prostatic urethra. In certain prior-art configurations, the massage can be concurrently performed with a thermal treatment.

Existing pressure-pulse-massage treatments for prostatitis require separate, dedicated components for: (1) creating and maintaining pressure; and (2) controlling certain variables—temperature, duration of treatment, mean catheter pressure, pulse amplitude, shape, and frequency. One example of a system that combines pressure-pulse-massage is described in International App. No. PCT/US02/0024221, which is incorporated herein by reference in its entirety. This reference discloses a closed-loop system to provide heated fluid, which is directed to a balloon-type catheter to provide thermal therapy of localized tissue in the prostatic urethra. The system includes a peristaltic pump for circulating fluid, maintaining a mean fluid pressure, and controlling circulation rate. A heater adjusts fluid temperature based on input received from sensors and linked to a controller, all of which may be operably associated with the catheter. The system can be configured as a low-volume system, circulating from between about 10-75 ml of fluid. The described system, however, does not provide for independent control of the fluid pulse-shape variables. Additionally, the system may require costly sterilization procedures prior to re-use or, alternatively, may be used only once.

In view of the above, there remains a need to provide improved and/or alternative treatments for chronic prostatitis.

### SUMMARY OF THE INVENTION

The present invention provides minimally invasive methods and systems for treating prostatitis by internally massaging a portion of the prostate. The internal massage can be carried out by repetitively outwardly expanding and then contracting an expandable treatment balloon held on the perimeter of a flexible translumenal catheter positioned in the prostatic urethra. In certain embodiments, the internal massage is directed to the portion of the prostate tissue located in the prostatic urethra (adjacent the bladder neck and above the verumontanum).

The pressure-pulse-massage treatment is typically provided by either a closed loop system or an open loop system. Each type of a system may include a single pump that directs pressurized fluid to a translumenal catheter, which is configured and sized for insertion in the urethra. The catheter further comprises an outwardly expandable treatment balloon. The treatment balloon is configured to repetitively expand and contract to provide a massage to the tissue located proximate thereto while the catheter circulates fluid via the expandable treatment balloon. Positive pressure may be created and maintained in the catheter by a flow restrictor located in the fluid return line.

The system includes a treatment set, or assembly, that may be coupled, or connected, to a catheter to provide treatment fluid thereto. The treatment set may include a fluid reservoir and a pump that directs fluid to and from the catheter through fluid supply and fluid return lines. Positive pressure in the catheter may be created and maintained by a flow restrictor in the fluid return line.

A user console may be removably connected, or coupled, to the treatment set to independently control pressure-pulse variables. The user console may include a motor controlled by an electronics system to drive the pump at variable speeds and a user interface to control the motor. In the system of the present invention, the pulse-shape variables can be controlled independently by cycling and varying the speed of the pump. Pressure-pulse variables, as used herein, comprise, but are not limited to any combination of the following: fluid pressure, temperature, duration, number of repetitive cycles, pulse shape, pulse frequency, pulse rate, pulse amplitude, circulation rate, and mean pulse, for example.

Independent operation of pressure-pulse variables, such as mean catheter pressure, pulse amplitude, shape, and frequency, may be obtained by precisely controlling the pump rotation speed. The rapid change of pump-motor speed can create pressure pulses that vary in amplitude, frequency, pressure, and rate. Cycling these pressure waves results in a pulse-massage in a downstream treatment device, such as a balloon catheter.

In one embodiment the motor is located in the control module and is coupled to a pump in the treatment set. The control module has a motor controller, a user-interface device, and motor-control electronics. Optionally, a drive transmitting mechanism couples the motor to the pump. The motor control electronics allow independent operation of at least one of the following: mean pulse, pulse amplitude, shape, or frequency of the fluid in the treatment device.

In one embodiment, the treatment set includes a pump in fluid communication with a treatment device. A flow restrictor is placed downstream from and adapted to maintain a positive pressure on the treatment device, such as a balloon catheter. One possible pump is a centrifugal pump. The treatment set includes a fluid reservoir upstream from and in fluid communication with the pump and a pressure-sense diaphragm upstream from the treatment device. In an alternative embodiment, the motor is located in the treatment set.

In certain embodiments, the invention is directed to a method of manufacturing a treatment system for treating prostatitis by providing a treatment device to apply therapeutic treatment to an organ of a living body by applying a fluid pressure-pulse therapy to an organ of a living body through pressure-pulse variables including at least one of fluid pressure pulse, pulse amplitude, pulse shape, and pulse frequency, providing a treatment set operably coupled to the treatment device to deliver treatment fluid to the treatment device, providing a flow restrictor associated with the treatment set for creating a positive pressure in the treatment device; and providing a control unit to independently control at least one of the pressure-pulse variables.

The system of the present invention overcomes limitations of the prior art and provides a system that reduces complexity, minimizes the number of components, reduces cost, and provides independent control of pulse-shape variables, circulation rate and pressure.

The foregoing and other objects and aspects of the present invention are explained in detail in the specification set forth below.

### BRIEF DESCRIPTION OF THE DRAWINGS

FIG. 1 is a schematic section view illustrating a catheter with an expandable treatment balloon in position in the prostatic urethra according to embodiments of the present invention.

FIG. 2 is an enlarged top view of the catheter shown in FIG. 1, illustrating the expandable balloon taking on expanded and contracted shapes according to the arrows illustrated in FIG. 1.

FIG. 3 is a block diagram of a method of treating prostatitis according to embodiments of the present invention.

FIG. 4 is a schematic illustration of a closed-loop fluid circulating system according to embodiments of the present invention.

FIG. 5 is a schematic illustration of an open-loop fluid circulating system according to embodiments of the present invention.

FIG. 6 is a detail sectional view of the flow restrictor of FIGS. 4 and 5.

FIG. 7 is a schematic illustration of an alternative embodiment of the present invention.

### DESCRIPTION OF EMBODIMENTS OF THE INVENTION

The present invention will now be described more fully hereinafter with reference to the accompanying figures, in which preferred embodiments of the invention are shown. This invention may, however, be embodied in many different forms and should not be construed as limited to the embodiments set forth herein. Like numbers refer to like elements throughout. In the figures, certain components, features or layers may be exaggerated for clarity. In the block diagrams, broken lines indicate optional features or steps.

The present invention is directed at methods and devices for treating diseases of the prostate and may be particularly suitable for treating chronic prostatitis (such as Type II, III or IV, and more particularly the Type III or IV). However, using the prostate as a model, and the teachings herein, persons skilled in the art will understand how the invention may be therapeutically used to treat other anatomical features.

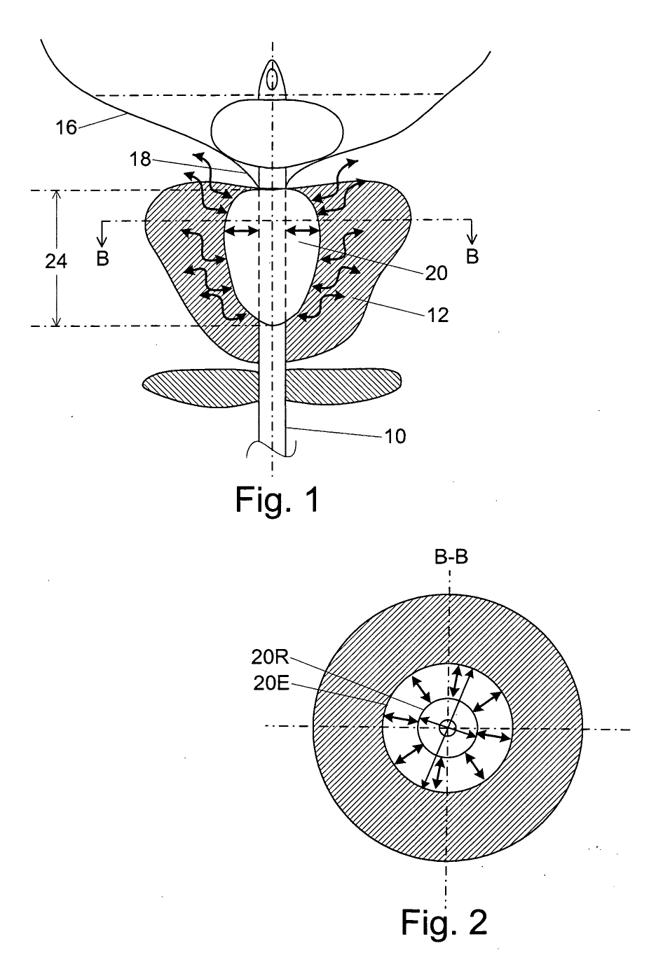

FIG. 1 illustrates a trans-luminal elongated catheter 22, which may be inserted into the prostatic urethra 10 via the penile meatus and through the male urethra. The catheter 22 can be a flexible catheter so as to be able to be inserted into position with minimal patient discomfort. See, e.g., U.S. Pat. Nos. 5,257,977, 5,549,559, and 5,084,044; and U.S. Provisional Patent Application Ser. Nos. 60/248,109, and 60/288,774, the contents of these documents are hereby incorporated by reference as if recited in full herein.

At least one treatment balloon 20 is positioned on an outer perimeter of catheter 22. The treatment balloon 20 expands outwardly when in position, and is adapted to contact localized tissue in the prostatic urethra 24. Although shown throughout as a single treatment balloon positioned on a distal portion of the catheter, other configurations can also be employed. For example, the internal massage can be administered using a plurality of circumferentially spaced balloons or axially spaced balloons.

In operation, the treatment balloon 20 is configured to repetitively outwardly expand and then contract a desired distance (illustrated by the bi-directional lateral or horizontal arrows in FIG. 1). In certain embodiments, the treatment balloon 20 expands and remains in contact with the localized tissue (or wall of the lumen) during both the expansion and contraction cycles.

FIG. 2 shows a possible treatment balloon that is partially contracted to a smaller cross sectional width (shown as diameter D1) to ease the massage or contact force (or pressure) applied to the prostate. In comparison, an increased force or pressure applied by a larger expanded shape (corresponding to the larger cross-sectional width) is shown as D2. Thus, the “Δp”, or change in massage pressure, results from the change in the expansion of the treatment balloon “ΔD”.

In certain embodiments, in situ, the treatment balloon 20 may not actually change its shape to the same extent when positioned inside the body as compared to when it is outside the human body of the patient. That is, the expansion movement of the balloon may be inhibited or blocked by the lumen of the prostate (and/or the resiliency of the underlying prostatic tissue). However, when operated outside the body, the treatment balloon will either visibly alter its shape, provide a pulsatile feeling when held, or both. During the internal massage the prostatic tissue may yield (become more elastic), allowing the balloon 20 to increase in size during the internal massage. Alternatively, or in addition, the balloon may be intentionally expanded to provide an increased massage force or pressure. For the latter, the balloon 20 may substantially retain its pre-increased force or pressure configuration (as it is encased by the density and shape of the adjacent prostatic tissue) but still provide the increased massage force to the adjacent tissue (to promote penetration depth).

The treatment balloon 20 can be configured so that it can apply a substantially constant circumferentially distributed massage (the circumferentially distributed massage is represented by the bi-directional arrows about the outer surface of the balloon) over at least a portion of, and preferably a major length of, the prostatic urethra. This can provide a substantially uniform distribution of a massage around the urethra to increase the likelihood that the treatment will have sufficient penetration so as to positively impact the prostate capsule. The balloon 20 can be inflated with any suitable (preferably biocompatible) inflation medium.

In certain embodiments, the fluid may be circulated to cause the balloon 20 to perform the internal massage. This fluid may be a biocompatible fluid, such as water, or an aqueous formulation. Fluid may be circulated at any temperature: non-elevated, heated, or cooled. In a preferred embodiment a treatment fluid at a non-elevated temperature is circulated and used to massage the local tissue prior to, after, or concurrent with applying an alternative therapy type. The alternative therapy can be one, or a combination of, an oral therapeutic agent, locally delivered therapeutic agent, or other therapy type, such as radiation, chemotherapy, or thermal therapy.

The term therapeutic agent includes medicines, food supplements, or bioactive substances or formulations used to treat prostatitis or its symptoms (delivered either systemically or locally during or as an adjunct to the massage therapy), including over the counter or prescription pharmaceutical products, vitamins or food, beta radiation, and the like.

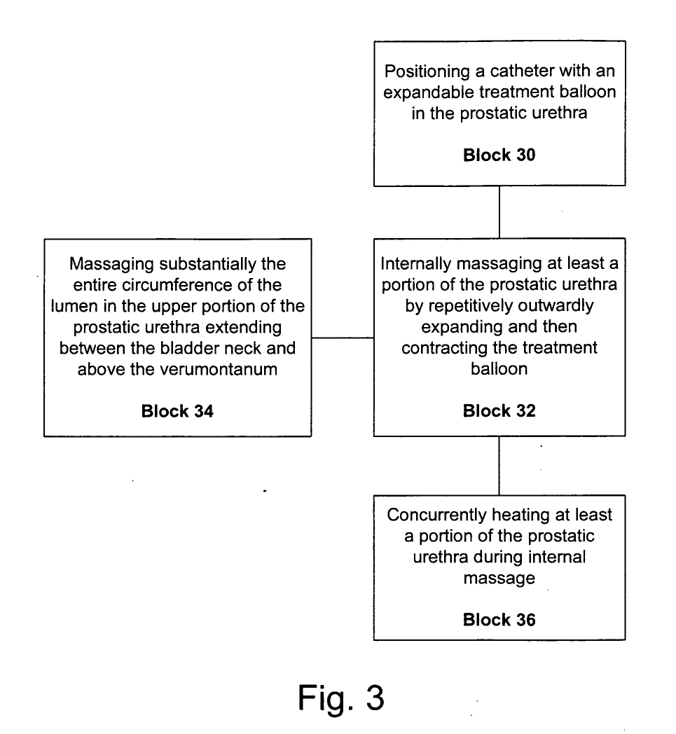

FIG. 3 shows a system for treating prostatitis consisting of inserting, or positioning, a catheter with an expandable treatment balloon in the prostatic urethra (block 30) and internally massaging at least a portion of the prostatic urethra by repetitively outwardly expanding and contracting the treatment balloon (block 32). The method can be carried out so that substantially the entire perimeter of the lumen adjacent the treatment balloon (shown as circumferentially administered) is massaged, preferably substantially the entire region of the prostatic urethra (extending between the bladder neck and the verumontanum) (block 34).

In certain embodiments, the massage-pulse variables (such as intensity or frequency) can be altered during the treatment.

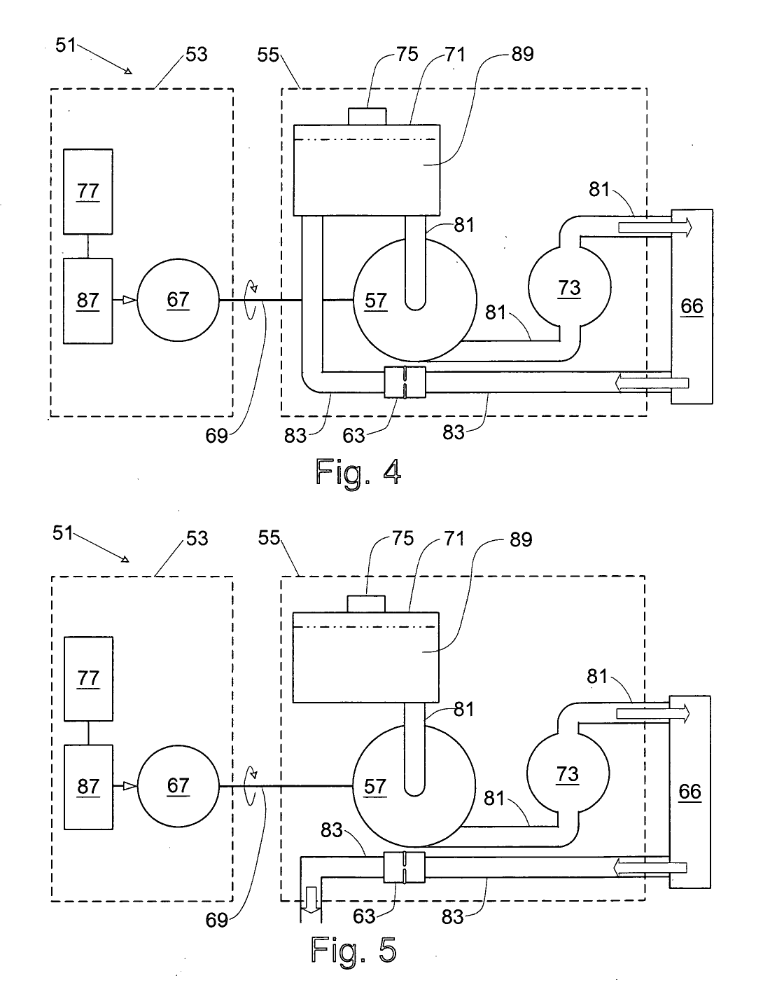

In closed loop fluid circulating systems (shown in FIG. 4, for example), the magnitude of the massage force or pressure can be increased (or decreased) over the treatment period, by adjusting the flow rate or volume (or pressure) of the fluid in the system and, thus, the fluid pressure in (the degree of expansion of) the treatment balloon. See e.g., U.S. patent application Ser. No. 09/433,952 and U.S. Pat. No. 5,549,559, the contents of which are hereby incorporated by reference as if recited in full herein, for descriptions of a suitable closed loop circulating fluid system. The pressure may actually be measured and any correction may be carried out by the electronics used to drive the motor. The pressure in the treatment balloon (which corresponds to the pressure in the closed loop fluid circulating system) may be from about 0.5-1.5 atm, and typically at least about 1 atm during at least a portion of the treatment to increase the pulsation force presented to the localized tissue.

FIG. 4 illustrates one embodiment of a system 51 for treating prostatitis, for example. The system includes a treatment set 55 and a control module 53 that delivers pulsating fluid to a treatment device 66, such as a catheter. Treatment set 55 includes a pump 57 that supplies treatment fluid to a treatment device 66 from a fluid reservoir 71 through a fluid supply line 81. Fluid reservoir 71 has an optional air-purge element 75 to remove any gaseous matter that is undesirably trapped in the fluid 89. Pump 57 may be a centrifugal pump. However, many types of pumps may work equally well. A single pump 57 maintains positive pressure via a flow restrictor 63 and independently controls the fluid pulse-shape. Because there is only one pump 57, there is cost savings, reduced complexity, and improved efficiency. Fluid flows through the treatment device 66 and returns to treatment set 55 through a fluid return line 83. A pressure-sensing diaphragm 73 is optionally provided between pump 57 and treatment device 66 to sense the fluid pressure in supply line 81 and communicate the pressure to control electronics. In an alternative embodiment (not shown), pressure-sensing diaphragm 73 is electronically connected to control module 53 to vary and control the fluid pressure. In this embodiment, pressure-sensing diaphragm 73 senses fluid pressure and electronically communicates with control module 53, which may then vary the motor speed to vary and control the fluid pressure.

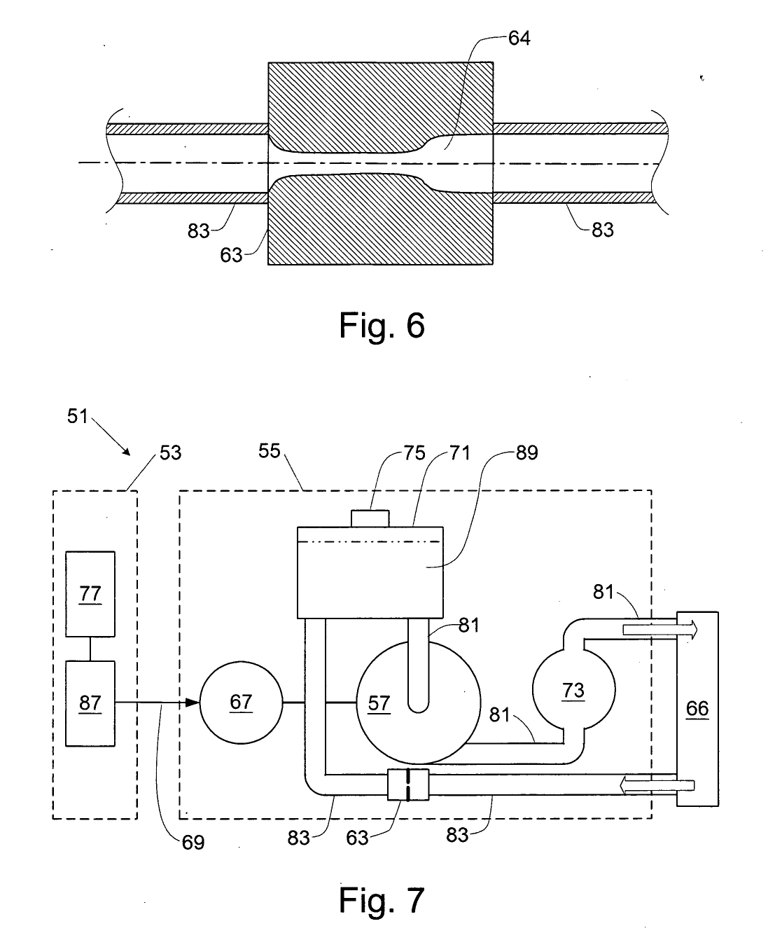

To provide positive pressure to treatment device 66, flow restrictor 63 may be located in fluid communication with return line 83 downstream from treatment device 66. The embodiment of FIG. 6 shows one type of flow restrictor 63 that creates positive pressure in treatment device 66 by narrowing the cross-section of the internal diameter of the fluid-flow path. Flow restrictor 63 of FIG. 6 has a substantially narrower fluid cross section 64 than return line 83. Alternatively, the flow restrictor may include a diaphragm with a small-diameter hole for constraining fluid flow, a flow valve, or any other device that creates positive pressure in the treatment device. Additionally, a flow restrictor may be a small diameter return line 83.

In this embodiment, control module 53 is remotely located and detachable from treatment set 55. A drive transmission mechanism 69 couples control module 53 to treatment set 55. As used herein, the terms couple, coupling, and coupled define a permanent, semi-permanent, temporary, releasable, or selective engagement between two adjacent elements.

In one embodiment, drive transmission mechanism 69 is operably coupled to and in driving engagement with pump 57 from motor 67 located in control module 53. Drive transmission mechanism 69 provides drive motion between remotely-located motor 67 in control module 53 and pump 57. Treatment set 55, control module 53, or both may include an interface device, such as an outlet, plug, or receptacle, for example, for coupling to drive transmission mechanism 69. Drive transmission mechanism 69 also may be an assembly of individual linking elements coupled together including an intermediate interface device or plurality of such devices. Furthermore, drive transmission mechanism 69 may be coupled directly to both control module 53 and treatment set 55.

Control module 53 includes a user-interface 77, which includes an input device for entering user-controllable variables including pulse-shape variables, duration of the treatment, and treatment fluid temperature. The pulse-shape variables include mean catheter-pressure, pulse amplitude, pulse frequency, and circulation rate. The input device communicates with motor-control electronics 87 for providing input to pump 57 for independent operation of all the variables. An optional memory device (not shown) is used to store previously programmed parameter profiles. The user can select from the profiles to control the variables in any combination and in real-time.

Motor 67 may be an electric, variables-speed motor for controlling the fluid pressure. For example, as the motor speed increases, pump 57 directs a greater volume of fluid 89 to treatment device 66. This results in an increase in backpressure created by flow restrictor 63. Thus, by altering the pump speed, fluid pressure can be varied. Furthermore, rapidly changing the motor speed creates the pressure-pulses and associated variables used in the therapeutic treatment. In the closed loop fluid circulation system of FIG. 4, the actual pressure is measured and precise control is maintained by electronics, for example, motor controller 87. Treatment set 55 may be disposable and control module 53 may be reusable. Because treatment fluid does not interact with re-usable control module 53, expensive and hazardous sterilization procedures are not required. To initiate a new treatment procedure, a new treatment set simply may be coupled to re-usable module 53.

FIG. 5 shows an open-loop fluid circulation system 51 of the present invention. As used herein, an open-loop fluid circulation system draws treatment fluid 89 from a first source, such as a reservoir 71, and discharges fluid without returning the fluid to the first source. With this in mind, the open-loop fluid circulation system operates the same as a closed loop fluid circulation system. Fluid pressure, however, is measured and controlled from the motor speed setting and treatment fluid 89 is discharged from treatment device 61 without recycling to pump 57.

FIG. 7 shows an alternative embodiment of the present invention wherein pump 57 and motor 67 are both contained in treatment set 55. In this example, drive transmission mechanism 69 relays commands from motor controller 87 to motor 67, which is integrated with treatment set 55. Otherwise, this embodiment operates similar to the embodiment of FIG. 4. In this embodiment, treatment set 55, including motor 67, is disposable.

Thus, the internal massage is provided by cycling the speed of pump 57 to generate pulsatile fluid flow. Other methods for expanding and contracting a treatment balloon or generating the pulsatile flow can also be used as will be appreciated by those of skill in the art. In certain embodiments, the pulstatile flow can be provided so that the expansion and contraction rate at the treatment balloon is about 1-12 pulses per second or more, and typically about 1-5 pulses per second. This action can be used to alter the delivered massage force by causing the treatment balloon 20 to repetitively expand and contract during at least a portion of the treatment, and preferably during the entire treatment session.

As shown in FIG. 1, in certain embodiments, treatment balloon 20 is configured with an axial length selected to resides above the vemmontanum of the patient. As shown, one end of treatment balloon 20 extends above, or proximate to, the bladder-neck of the patient and the other end extends proximate to the verumontanum. Thus, for the treatment of prostatitis, the treatment balloons may be modified from the WIT™ catheters used in the Thermoflex® System (ACMI Corporation, Southborough, Mass.) for treating BPH. That is, as shown in FIG. 4, the catheters 20 may be configured in an array of different treatment balloon 20 sizes, lengths, or both to provide a custom fit for the patient. Further, in contrast to catheters with treatment balloons used to treat BPH (typically having lengths from about 2-6 cm), it may be desirable to configure the treatment balloons so that they have shorter axial lengths. In certain embodiments, the catheters 20 are produced in sets or in increments of from about 1-6 cm. Additionally, catheter 22 may be configured with a bladder-anchoring balloon (similar to a Foley-type catheter) located above the treatment balloon 20 to secure the treatment balloon in position during the treatment. In certain embodiments, the treatment balloon 20 may be extended a length sufficient to allow an upper portion to reside adjacent the bladder neck to allow the bladder neck to be thermally treated concurrently with the prostatic urethra.

In certain embodiments, the massage force, the repetition rate of expansion and contraction, or both may be altered on a customized level to optimize patient comfort. For example, the massage force or pressure in the system may be increased over time as the prostatic tissue becomes more yielding or the patient less sensitive to the force in the prostatic urethra. The application of pressure may provide increased therapeutic responsiveness over thermo therapies alone.

In addition, the massage treatment can be used while administering a therapeutic agent or radiation. For example, the treatment fluid may also comprise a therapeutic agent and the treatment device may include a flowable inflation medium. For example, the therapeutic agent could permeate the treatment balloon during the massage and migrate into the tissue. The therapeutic agent may be configured to maintain its efficacy at elevated temperatures (at least between about 40-43° C.). In other embodiments the therapeutic agent can be applied as a coating on the perimeter surface of the balloon so that the tissue is exposed upon contact with the balloon during the treatment. In still other embodiments, a portion of the catheter such as the external surface of the expandable treatment balloon can be coated with a radioisotope of beta emitting isotope (such as P(32), Y(90), or Sr(90)).

In certain embodiments, a single treatment session may be sufficient to treat prostatitis. In other embodiments a plurality of successive treatments performed over a treatment window (such as over 1 week, 1 month or 1 quarter of a calendar year) is required. The massage-pulse therapy may also be combined with other desired supplemental or adjunct treatment regimens such as lifestyle changes, exercise, and/or selected therapeutic agents or food supplements (alone, or in combination). The desired supplemental treatment may be initiated prior to, during or after the pulse-massage treatment(s) and may also continue for periods of time thereafter. Examples of therapeutic agents which may be beneficial include one or more of analgesics, anti-depressants, phytotherapy therapeutics such as PEENUTS or PROSTA-Q, anti-inflammatory agents such as steroid inhibitors (such as COX-2 inhibitors like VIOXX) and PENTOSAN POLYSULFATE, non-steroid inhibitors, antibiotics, neuroleptic agents (such as ELVAIL, NEURONTIN, DOXEPIN, and MARCAINE), a-blockers (such as PHENOXIBENZAMINE), specific immunology modulators such as ENBREL (by Immunex, a drug approved by the FDA for rheumatoid arthritis, an autoimmune disease), biofiavinoids to reduce the level or oxidants in the prostatic fluid, FINASTERIDE, antioxidants, quercitan, and the like.

The foregoing is illustrative of the present invention and is not to be construed as limiting. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. The invention is defined by the following claims, with equivalents of the claims to be included therein.

1. A system for providing fluid dynamics in a medical procedure, comprising: a treatment device for applying therapeutic treatment to an organ of a living body through pressure-pulse variables, a treatment set including a fluid reservoir, a pump coupled to the fluid reservoir, a supply line for delivering fluid from the pump to the treatment device, and a device associated with the supply line for creating positive pressure in the treatment device, and a control module coupled to the treatment set to independently control the pressure-pulse variables.

2. A system for providing fluid dynamics in a medical procedure, comprising:
a treatment device for applying therapeutic treatment to an organ of a living body, a treatment set coupled to the treatment device, the treatment set including a fluid reservoir and a pump in fluid communication with the treatment device, and a flow restrictor adapted to provide positive fluid pressure in the treatment device, and a control module coupled to the treatment set to control the fluid pressure in the treatment device.

3. A system for providing fluid dynamics in a medical procedure, comprising:
a treatment device for applying therapeutic treatment to an organ of a living body through pressure-pulse variables,
a treatment set coupled to the motor controller, the treatment set including a pump in fluid communication with the treatment device and a flow restrictor to create and maintain a positive pressure in the treatment device, and a control module operatively coupled to the pump including a motor and a motor controller for controlling the pump.

4. The system of claim 3 wherein the treatment device is a balloon catheter.

5. The system of claim 3 wherein the pump is a centrifugal pump.

6. The system of claim 3 wherein the motor is an electric motor operatively to the pump by a drive transmitting mechanism.

7. The system of claim 3 wherein the motor is controlled to independently vary the pressure-pulse variables.

8. The system of claim 3 wherein the treatment set further comprises a fluid reservoir in fluid communication with the pump to supply fluid to the treatment device.

9. The system of claim 3 wherein the treatment set further comprises a pressure-sensing diaphragm upstream from the treatment device.

10. The system of claim 3 wherein the control module further comprises a user-interface device.

11. The system of claim 10 wherein the user-interface device provides input to the pump for independent operation of at least one of temperature, mean pulse, pulse amplitude, shape, or frequency of the fluid in the treatment device.

12. The system of claim 3 wherein the control module further comprises motor-control electronics for providing input to the pump for independent operation of at least one of temperature, mean pulse, pulse amplitude, shape, or frequency of the fluid in the treatment device.

13. A treatment catheter system, comprising:
a treatment catheter for applying a pressure-pulse therapy to an organ of a living body through pressure-pulse variables including at least one of fluid pressure pulse, pulse amplitude, pulse shape, and pulse frequency, a treatment set including a pump in fluid communication with a treatment catheter and a flow restrictor associated with the pump to provide positive pressure to the treatment catheter, and a control module operatively coupled to the pump to independently control at least one of the pressure-pulse variables.

14. The treatment catheter system of claim 13, wherein the control module further comprises a motor removably coupled to the pump through a transmitting mechanism, and a motor controller to control the motor.

15. The treatment catheter system of claim 14, wherein the motor comprises an electric motor and the transmitting mechanism comprises a removable shaft for driving the pump.

16. The treatment catheter system of claim 13, wherein the treatment set further comprises a motor for driving the pump.

17. The treatment catheter system of claim 13, wherein the treatment set further comprises a fluid reservoir upstream from and in fluid communication with the pump.

18. The treatment catheter system of claim 13, wherein the treatment set further comprises a pressure-sensing diaphragm upstream from the treatment device.

19. The treatment catheter system of claim 13, wherein the control module further comprises a user-interface device.

20. The treatment catheter system of claim 19, wherein the user-interface device provides input to the pump for independent operation of at least one of the fluid pressure pulse, pulse amplitude, pulse shape, and pulse frequency in the treatment device.

21. The treatment catheter system of claim 13, wherein the control module further comprises motor-control electronics for providing input to the pump for independent operation of at least one of the fluid pressure pulse, pulse amplitude, pulse shape, and pulse frequency in the treatment device.

22. A prostatic treatment system comprising:
a catheter system including a treatment catheter, a treatment set including a pump for directing fluid to the catheter through a fluid supply line, the fluid supply line having an associated flow restrictor for creating a positive pressure in the treatment catheter, and a user console operatively coupled to the catheter system for remote operation of the catheter system, the user console including a user interface and a controller for controlling the pump.

23. The prostatic system of claim 22, further comprising a transmitting mechanism operably coupled between the user console and the pump to drive the pump.

24. The prostatic system of claim 23, wherein the user console further comprises an electric motor operated by the controller, and wherein the transmitting mechanism comprises a removable shaft for driving the pump.

25. The prostatic system of claim 22, wherein the treatment set further comprises a motor for driving the pump.

26. The prostatic system of claim 22, wherein the treatment set further comprises a fluid reservoir upstream from and in fluid communication with the pump.

27. The prostatic system of claim 22, wherein the treatment set further comprises a pressure sensing diaphragm upstream from the treatment device.

28. The prostatic system of claim 22, wherein the user console further comprises a user-interface device.

29. The prostatic system of claim 28, wherein the user-interface device provides input to the pump for independent operation of at least one of fluid pressure pulse, pulse amplitude, pulse shape, and pulse frequency in the treatment device.

30. The prostatic system of claim 22, wherein the user console further comprises motor-control electronics for providing input to the pump for independent operation of at least one of fluid pressure pulse, pulse amplitude, pulse shape, and pulse frequency in the treatment device.

31. A method of manufacturing a treatment system for treating prostatitis, comprising:
providing a treatment device for applying a pressure-pulse therapy to an organ of a living body through pressure-pulse variables including at least one of fluid pressure pulse, pulse amplitude, pulse shape, and pulse frequency, and
providing a user console operatively coupled to the treatment device to independently control at least one of the pressure-pulse variables.

32. A method of manufacturing a treatment system for treating prostatitis, comprising;
providing a treatment device to apply therapeutic treatment to an organ of a living body by applying a fluid pressure-pulse therapy to an organ of a living body through pressure-pulse variables including at least one of fluid pressure pulse, pulse amplitude, pulse shape, and pulse frequency, providing a treatment set operably coupled to the treatment device to deliver treatment fluid to the treatment device, providing a flow restrictor associated with the treatment set for creating a positive pressure in the treatment device; and providing a control unit to independently control at least one of the pressure-pulse variables.

33. The method of claim 32, further providing a closed-loop treatment set.

34. The method of claim 32, further providing an open-loop treatment set.

35. The method of claim 32, further providing a transmission device between the control unit and the treatment set.
